SEQUENCE LISTING (6 sequences)

SEQ ID NO: 1 - protein, 15 aa, Homo sapiens
TSYVKVLHHMVKISG

SEQ ID NO: 2 - protein, 14 aa, Homo sapiens
LLKYRAREPVTKAE

SEQ ID NO: 3 - protein, 21 aa, Homo sapiens [sequence omitted]

SEQ ID NO: 4 - protein, 23 aa, Homo sapiens
RNGYRALMDKSLHVGTQCALTRR

SEQ ID NO: 5 - protein, 16 aa, Homo sapiens
WNRQLYPEWTEAQRLD

SEQ ID NO: 6 - protein, 16 aa, Homo sapiens
AQNILLSNAPLGPQFP

(19) United States
(12) Patent Application Publication
(10) Pub. No.: US 2025/0269003 A1
(43) Pub. Date: Aug. 28, 2025

(54) NANO-ENHANCED VACCINE

(71) Applicant: University of Virginia Patent Foundation, Charlottesville, VA (US)

(72) Inventors: Mark Kester, Afton, VA (US); Craig L. Slingluff, Charlottesville, VA (US); Helena Woodvine Snyder, Crozet, VA (US); Katelyn Salotto, Lebanon, NH (US); Elishama Michel, Leesburg, VA (US)

(73) Assignee: University of Virginia Patent Foundation, Charlottesville, VA (US)

(21) Appl. No.: 18/859,144

(22) PCT Filed: Apr. 24, 2023
(86) PCT No.: PCT/US2023/066134

§ 371 Date: Oct. 22, 2024

Related U.S. Application Data
(60) Provisional application No. 63/334,027, filed on Apr. 22, 2022.

(51) Int. Cl. A61K 39/00; A61P 35/00

(52) U.S. Cl. CPC A61K 39/001191; A61K 39/001156; A61K 39/001186; A61K 39/001192; A61P 35/00; A61K 2039/54; A61K 2039/55555

(57) ABSTRACT

Provided are composition that include stable TLR4 agonist (e.g., KDO2) containing nanoliposomes. In some embodiments, the TLR4 agonist (e.g., KDO2) containing nanoliposome include a lipid component comprising, consisting essentially of, or consisting of DSPC, DOPE, PEG(2000)-PE, one or more TLR4 agonists (e.g., KDO2), Cholesterol, Rhodamine or DiD, and optionally DOTAP and/or DHP. In some embodiments, the TLR4 agonist (e.g., KDO2) containing nanoliposomes are cationic, anionic, or neutral liposomes. In some embodiments, the TLR4 agonist (e.g., KDO2) containing nanoliposome encapsulate one or more immunogenic peptides, which can be peptides associated with malignant melanoma, which optionally can be subsequences of tyrosinase, gplOO, MAGE-1,2,3,6, Melan-A/MART-1, and/or MAGE-3. Also provided are methods for treating and/or preventing malignant melanoma and for inducing anti-melanoma immune responses in subjects using the presently disclosed compositions.

### CROSS REFERENCE TO RELATED APPLICATIONS

The presently disclosed subject matter claims priority to and the benefit of U.S. Provisional Patent Application Ser. No. 63/334,027, filed Apr. 22, 2022, the disclosure of which is herein incorporated by reference in its entirety.

### GOVERNMENT INTEREST

This invention was made with government support under Grant No. CA044579 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 6,086 byte UTF-8-encoded XML file created on Apr. 24, 2023 and entitled “3062_187_PCT.xml”. The Sequence Listing submitted via Patent Center is hereby incorporated by reference in its entirety.

### TECHNICAL FIELD

The presently disclosed subject matter relates generally to compositions comprising KDO2-containing nanoliposomes and methods for using the same to treat and/or prevent malignant melanoma and/or for inducing immune responses against malignant melanoma.

### BACKGROUND

Melanoma of the skin is the 5^{th} most common cancer for both males and females in the U.S. (Siegel et al., 2021) and currently, only complete surgical removal is considered curative. In advanced malignant and metastasized stages of melanoma, where surgery alone cannot offer remission, prognosis is extremely poor. This is because human melanoma is particularly resistant to conventional chemotherapies and other cytotoxic drugs (Grossman & Altieri, 2001; La Porta, 2007; Winder & Virós, 2018) and this resistance has primarily been linked to dysregulations in apoptosis (Grossman & Altieri, 2001).

Recent progress in cancer immunotherapy has prolonged median survival for metastatic melanoma from about 9 months to about 3 years, but most patients with metastatic melanoma still do not survive (Rudmann, 2013; Dance, 2017; Lim et al., 2019). A lack of pre-existing T cell immunity to cancer antigens is commonly a root cause of failure. In addition, both intrinsic and adaptive resistance to immunotherapies in melanoma has been observed (Winder & Virós, 2018). Therefore, there is a significant need for new therapies to prevent melanoma recurrence by enhancing immune responses to melanoma. Cancer vaccines offer the promise to induce immune responses to cancer when spontaneous antitumor immunity is absent or weak. The effectiveness of immune checkpoint blockade therapy demonstrates that melanoma cells express antigens that can be recognized by CD4 and CD8 T cells, and also that T cell responses to those antigens can mediate tumor regression, when tumor-associated immune dysfunction is abrogated. There has been recent enthusiasm for approaches targeting antigens created by spontaneous mutations (mutated neoantigens; Blass & Ott, 2021) however, recent data highlight the value of shared melanocytic antigens as relevant targets (Lo et al., 2021) and other data highlight the value of vaccinating against shared cancer-testis antigens (Suri, 2006).

Data from various groups have highlighted the pivotal role of CD4⁺ (helper) T cells in cancer immunity and cancer control (Hunder et al., 2008; Slingluff et al., 2010; Friedman et al., 2012; Kitano et al., 2013). A multi-peptide melanoma vaccine consisting of six melanoma helper peptides (6MHP) designed to induce melanoma-reactive CD4⁺ T cells has been previously described, and it was found that they were immunogenic in humans, inducing objective clinical responses and very high rates of 5-year survival. It was additionally found that the immune responses were enhanced when simultaneously injecting a toll like receptor 3 (TLR3) agonist with the vaccine (Slingluff et al., 2021). It was also found that, with a different peptide vaccine, agonists for TLR4 are useful vaccine adjuvants alone, or with incomplete Freund's adjuvant (Melssen et al., 2019). However, these responses are still modest in absolute magnitude even with strong adjuvants, and even the best responses usually represent less than 1% of circulating CD4⁺ T cells. A major goal of current cancer vaccines is to induce stronger and durable T cell responses, but optimal vaccine strategies in humans have not yet been defined.

Most cancer vaccines contain antigens plus vaccine adjuvants, but when they are co-administered as soluble agents, there is no assurance that the antigens and the adjuvants will be delivered to the same antigen-presenting cell. Nanoliposomes provide a way to deliver antigens to phagocytic antigen-presenting cells, especially dendritic cells, while simultaneously delivering vaccine adjuvants to the same antigen-presenting cells. The use of nanoliposomes for the delivery of cancer therapeutics offers significant promise and has had several clinical successes (Gabizon et al., 2003). Indeed, many vaccines benefit from lipid-based delivery. Of particular note are the recently authorized COVID-19 vaccines, which utilize lipid nanoparticles containing antigen-encoding RNA (Chung et al., 2020; Pacheco et al., 2020). The present co-inventors have been successful in the use of nanotechnologies, including nanoliposomes, for a host of different therapies (Li et al., 2018; Shin et al., 2018; Zhang et al., 2018; Barth et al., 2019; Shaw et al., 2020; Zanieri et al., 2020). Nanoliposomes have the capability of altering the pharmacokinetic properties of antigens and immunomodulators/adjuvants, with a marked reduction in off-target toxicity, protection of the therapeutic from degradation, along with more specific targeting to antigen-presenting cells (Nisini et al., 2018). A theoretical benefit of using a nanoliposome vaccine over merely injecting antigens and adjuvant individually, is that the nanoliposome ensures all adjuvants circulate together and are delivered to the antigen presenting cells at the same time. Furthermore, nanoliposomes are readily taken up by the antigen-presenting dendritic cells (DCs; Fidler, 1988; Kelly et al., 2011; Ponzoni et al., 2018; Saremi et al., 2018); so, having a TLR agonist incorporated into the liposome should provide increased CD4⁺ T cell activation. Taken together, nanoliposomes are ideal nano-carriers and delivery agents for emerging cancer vaccines due to their versatility, ease of modification and ability to deliver several adjuvants simultaneously (Fan & Moon, 2015).

One of the advantages of liposomal delivery is the ability to incorporate vaccine adjuvants into the peptide delivery vehicle. Use of the toll-like-receptor 4 (TLR4) agonist, KDO2-Lipid A (herein KDO2, but also often referred to as KLA) is described herein. KDO2 is a synthetic and homogenous form of Lipid A, an essential component of lipopolysaccharides (LPS) in Gram-negative bacteria (Wang et al., 2015). KDO2 stimulates potent and reproducible host immune responses through the complex of Toll like-receptor 4 (TLR4) and myeloid differentiation protein 2 (Raetz et al., 2006; Sasaki & White, 2008; Wang et al., 2015). In previous studies, naturally-derived LPS was employed as a TLR4 agonist (Melssen et al., 2019) and others have used KDO2 and other Lipid A derivatives in several cancer immunotherapies (Livingston et al., 1985; Mata-Haro et al., 2007; Sims et al., 2010; Chiang et al., 2011); so, it is an appropriate choice of agonist for the presently disclosed subject matter. As described herein, the presently disclosed subject matter provides improve pharmacodynamics, pharmacokinetic and toxicological profiles by incorporating KDO2 directly into the delivery system to enhance CD4⁺ T cells via TLR4, in some embodiments by individually encapsulating each of six (6) melanoma helper peptides within their own immunogenic nanoliposomes. Thus, it was hypothesized that: (i) it would be possible to develop stable KDO2-containing nanoliposomes for the encapsulation of each of the six melanoma helper peptides identified previously; and (ii) these nanoliposomes would trigger an increased CD4⁺ T-cell response over and above delivery of the peptide(s) or TLR4 agonist alone.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to compositions comprising, consisting essentially of, or consisting of a stable nanoliposome containing one or more TLR4 agonists, optionally wherein at least one of the one or more TLR4 agonists is 3-deoxy-d-manno-octulosonic acid-lipid A (KDO2-lipid A or KDO2). In some embodiments, the KDO2-containing nanoliposome comprises a lipid component comprising, consisting essentially of, or consisting of one or more of, optionally two or more of, and further optionally all three of 1,2-distearoyl-sn-glycero-3-phosphocholine (DSPC); 1,2-Dioleoyl-sn-glycero-3-phosphoethanolamine (DOPE); and 1,2-distearoyl-sn-glycero-3-phosphoethanolamine-N-[methoxy(polyethylene glycol)-2000] (ammonium salt) (PEG2000PE), and optionally further comprises, consists essentially of, or consists of one or more of:
(i) a toll-like-receptor 4 (TLR4) agonist, optionally 3-deoxy-d-manno-octulosonic acid-lipid A (KDO2-lipid A or just KDO2); and/or
(ii) a rigidity enhancer and/or leakiness reducer, optionally cholesterol; and/or
(iii) a dye, optionally a fluorophore, further optionally rhodamine or 1,1′-Dioctadecyl-3,3,3′,3′-Tetramethylindodicarbocyanine, 4-Chlorobenzenesulfonate Salt (DiD); and/or
(iv) optionally 1,2-dioleoyl-3-trimethylammonium-propane (chloride salt) (DOTAP), dihexadecyl phosphate (DHP), or a combination thereof, or any combination thereof.
   In some embodiments, the KDO2-containing nanoliposome is a neutral liposome comprising a lipid component comprising, consisting essentially of, of consisting of DSPC, DOPE, PEG(2000)-PE, KDO2, Cholesterol, and Rhodamine or DiD at a molar ratio of about 4.6:1.14:0.25:0.01:3.00:0.02, optionally in a buffer comprising about 1.33 mg/ml sodium bicarbonate in a 1:2 solution of Lactated Ringers solution (LR) and water. In some embodiments, the KDO2-containing nanoliposome is a cationic liposome comprising a lipid component comprising, consisting essentially of, of consisting of DSPC, DOPE, PEG(2000)-PE, KDO2, Cholesterol, Rhodamine or DiD, and DOTAP at a molar ratio of about 4.12:1.90:0.25:0.01:3.00:0.02:0.70, optionally in a buffer comprising about 5 mg/ml sodium bicarbonate in water. In some embodiments, the KDO2-containing nanoliposome is an anionic liposome comprising a lipid component comprising, consisting essentially of, of consisting of DSPC, DOPE, PEG(2000)-PE, KDO2, Cholesterol, Rhodamine or DiD, and DHP at a molar ratio of about 3.91:1.81:0.25:0.01:3.00:0.02:1.00, optionally in a buffer comprising about 1 ml/ml sodium bicarbonate in water or a buffer comprising a 1:9 ratio of 2-(N-morpholino) methanesulfonic acid (MES) buffer and water.

In some embodiments of the presently disclosed subject matter, the KDO2-containing nanoliposome encapsulates an immunogenic peptide. In some embodiments, the immunogenic peptide is a peptide associated with malignant melanoma. In some embodiments, the peptide comprises, consists essentially of, or consists of an amino acid sequence that is a subsequence of a protein selected from the group consisting of tyrosinase, gp100, MAGE-1,2,3,6, Melan-A/MART-1, and MAGE-3. In some embodiments, the tyrosinase peptide comprises, consists essentially of, or consists of amino acids 56-70 (SEQ ID NO: 6) and/or 386-406 (SEQ ID NO: 3) of human tyrosinase; and/or the gp100 peptide comprises, consists essentially of, or consists of amino acids 44-59 (SEQ ID NO: 5) of human gp100; and/or the MAGE-1,2,3,6 peptide comprises, consists essentially of, or consists of amino acids 121-134 (SEQ ID NO: 2) of human MAGE-1,2,3,6; and/or the Melan-A/MART-1 peptide comprises, consists essentially of, or consists of amino acids 51-73 (SEQ ID NO: 4) of human Melan-A/MART-1; and/or the MAGE-3 peptide comprises, consists essentially of, or consists of amino acids 281-295 (SEQ ID NO: 1) of human MAGE-3. In some embodiments, the composition comprises 1, 2, 3, 4, 5, or 6 different peptides selected from the group consisting of a tyrosinase peptide, a gp100 peptide, a MAGE-1,2,3,6 peptide, a Melan-A/MART-1 peptide, and a MAGE-3 peptide, optionally wherein the tyrosinase peptide comprises, consists essentially of, or consists of SEQ ID NO: 3 or SEQ ID NO: 6; the gp100 peptide comprises, consists essentially of, or consists of SEQ ID NO: 5; the MAGE-1,2,3,6 peptide comprises, consists essentially of, or consists of SEQ ID NO: 2; the Melan-A/MART-1 peptide comprises, consists essentially of, or consists of SEQ ID NO: 4; and a MAGE-3 peptide comprises, consists essentially of, or consists of SEQ ID NO: 1.

In some embodiments, a composition of the presently disclosed subject matter is a pharmaceutical composition, optionally a pharmaceutical composition that is pharmaceutically acceptable for use in a mammal, further optionally wherein the mammal is a human.

In some embodiments, a composition of the presently disclosed subject matter further comprises, consists essentially of, or consists of at least one adjuvant. In some embodiments, the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

In some embodiments, the presently disclosed subject matter also relates to methods for treating and/or preventing malignant melanoma. In some embodiments, the methods comprise, consist essentially of, or consist of administering to a subject in need thereof an effective amount of a composition comprising, consisting essentially of, or consisting of a stable nanoliposome containing one or more TLR4 agonists, optionally wherein at least one of the one or more TLR4 agonists is 3-deoxy-d-manno-octulosonic acid-lipid A (KDO2-lipid A or KDO2). In some embodiments, the subject in need thereof is a human.

In some embodiments, the presently disclosed subject matter also relates to methods for inducing anti-melanoma immune responses in subjects. In some embodiments, the methods comprise, consist essentially of, or consist of administering to the subject an effective amount of a composition comprising, consisting essentially of, or consisting of a stable nanoliposome containing one or more TLR4 agonists, optionally wherein at least one of the one or more TLR4 agonists is 3-deoxy-d-manno-octulosonic acid-lipid A (KDO2-lipid A or KDO2). In some embodiments, the subject in need thereof is a human. In some embodiments, the administering is via a route selected from the group consisting of intravenous and subcutaneous.

The presently disclosed subject matter also relates to uses of compositions comprising a stable KDO2-containing nanoliposome encapsulating a tyrosinase peptide, and/or a gp100 peptide, and/or a MAGE-1,2,3,6 peptide, and/or a Melan-A/MART-1 peptide, and/or a MAGE-3 peptide, or any combination thereof, for treating and/or preventing malignant melanoma and/or for inducing an anti-melanoma immune response in a subject. In some embodiments, the KDO2-containing nanoliposome encapsulates at least one tyrosinase peptide, at least one gp100 peptide, at least one MAGE-1,2,3,6 peptide, at least one Melan-A/MART-1 peptide, and at least one MAGE-3 peptide. In some embodiments, the tyrosinase peptide comprises, consists essentially of, or consists of SEQ ID NO: 3 or SEQ ID NO: 6; the gp100 peptide comprises, consists essentially of, or consists of SEQ ID NO: 5; the MAGE-1,2,3,6 peptide comprises, consists essentially of, or consists of SEQ ID NO: 2; the Melan-A/MART-1 peptide comprises, consists essentially of, or consists of SEQ ID NO: 4; and a MAGE-3 peptide comprises, consists essentially of, or consists of SEQ ID NO: 1.

In some embodiments, the presently disclosed subject matter also relates to compositions comprising a stable KDO2-containing nanoliposome encapsulating a tyrosinase peptide, and/or a gp100 peptide, and/or a MAGE-1,2,3,6 peptide, and/or a Melan-A/MART-1 peptide, and/or a MAGE-3 peptide, or any combination thereof, for use in treating and/or preventing malignant melanoma and/or for inducing an anti-melanoma immune response in subjects.

Accordingly, it is an object of the presently disclosed subject matter to provide compositions comprising, consisting essentially of, or consisting of stable nanoliposomes containing one or more TLR4 agonists, optionally wherein at least one of the one or more TLR4 agonists is 3-deoxy-d-manno-octulosonic acid-lipid A (KDO2-lipid A or KDO2), and methods for use thereof, including but not limited to for treating and/or preventing malignant melanoma and/or for inducing an anti-melanoma immune response in subjects. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTION OF THE FIGURES

FIGS. 1A-1E. Typical dynamic light scattering data attained for each of the six melanoma helper peptides (FIG. 1A: AQN; FIG. 1B: WNR; FIG. 1C: LLK; FIG. 1D: FLL; FIG. 1E: RNG; and FIG. 1F: TSY). The data shows size distribution by intensity with size, d in nanometers along the x-axis and percentage intensity on the y axis. The graphs show a homogenous size distribution. The Z-average size (d·nm) attained from multiple samples is also provided for each peptide.

FIG. 2. Dynamic light scattering data attained after mixing all six peptide nanoliposome formulations together. The data shows size distribution by intensity, with size (d) in nanometers along the x-axis and percentage intensity on the y axis. The graphs show a homogenous size distribution. The Z-average size (d·nm) attained for this data is 113.5 nm.

FIG. 3. Fluorescent imaging of liposome biodistribution in mice on days 0 and 6. Two mice (SQ group) were injected subcutaneously in either flank and two mice (IV group) were injected intravenously.

FIG. 4. Showing the mean radiant efficiency in the extracted organs of four mice, two of which were injected with fluorescent and immunogenic liposomes via intravenous (IV) injection and two, who were administered the same nanoliposomes via subcutaneous (SQ) injection in either flank.

FIGS. 5A-5C. FIG. 5A. Graph of lymphocyte viabilities of Sentinel Immunized Nodes (SIN) post-treatment. Thick horizontal bars represent mean viability among treatments for a donor; FIG. 5B showing a graph of average proliferation of CD4⁺ cell populations after culture treatments with i) an empty anionic liposome, ii) an anionic liposome containing KDO2 but no peptide; iii) Free TSY (no liposome); iv) TSY encapsulated in an anionic liposome; and v) an anionic liposome containing TSY and KDO2; and FIG. 5C showing the 2D histograms used to calculate the data in FIG. 5B.

FIGS. 6A and 6B. FIG. 6A showing a schematic diagram of the unilamellar nanoliposome with the peptide dissolved within a stabilizing pH controlled buffer within the aqueous liposome core; cholesterol embedded within the lipod bilayer and a sparse PEG brush and immunogenic KDO2 lipid head group around the outer shell of the nanoliposome. The liposome is stored as a suspension 1×PBS. It should be noted that additional lipid components are added to create either a positive or negative charge in some of the formulations as. A fluorophore may also be added, which depending on the fluorophore used, may embed within the bilayer like cholesterol or be attached to a lipid and incorporate within the bilayer like PEG and KDO2. FIG. 6B shows the structure of KDO2-lipid A, which has 6 fatty acid chains and a head group on the surface.

### BRIEF DESCRIPTION OF THE SEQUENCE LISTING

SEQ ID NO: 1 is the amino acid sequence TSYVKVLHHMVKISG, which corresponds to amino acids 281-295 of the human MAGE-3 polypeptide (abbreviated MAGE-3₂₈₁₋₂₉₅) of Accession No. NP_005354.1 of the GENBANK® biosequence database. SEQ ID NO: 1 is also abbreviated herein as “TSY”.

SEQ ID NO: 2 is the amino acid sequence LLKYRAREPVTKAE, which corresponds to amino acids 121-134 of the human MAGE 2, MAGE 3, and MAGE 6 polypeptide and amino acids 114-127 of the human MAGE 1 polypeptide (abbreviated MAGE-1,2,3,6₁₂₁₋₁₃₄), Accession Nos. NP_001269430.1, NP_005353.1, NP_005354.1, and NP_004979.3 of the GENBANK® biosequence database, respectively. SEQ ID NO: 2 is also abbreviated herein as “LLK”.

SEQ ID NO: 3 is the amino acid sequence FLLHHAFVDSIFEQWLQRHRP, which corresponds to amino acids 386-406 of the human tyrosinase polypeptide (abbreviated Tyrosinase₃₈₆₋₄₀₆) of Accession No. NP_000363.1 of the GENBANK® biosequence database. SEQ ID NO: 3 is also abbreviated herein as “FFL”.

SEQ ID NO: 4 is the amino acid sequence RNGYRALMDKSLHVGTQCALTRR, which corresponds to amino acids 51-73 of the human Melan-A/MART-1 polypeptide (abbreviated Melan-A/MART-1₅₁₋₇₃) of Accession No. NP_005502.1 of the GENBANK® biosequence database. SEQ ID NO: 4 is also abbreviated herein as “RNG”.

SEQ ID NO: 5 is the amino acid sequence WNRQLYPEWTEAQRLD, which corresponds to amino acids 44-59 of the human gp100 polypeptide (abbreviated gp100₄₄₋₅₉) of Accession No. NP_001186983.1 of the GENBANK® biosequence database. SEQ ID NO: 5 is also abbreviated herein as “WNR”.

SEQ ID NO: 6 is the amino acid sequence AQNILLSNAPLGPQFP, which corresponds to amino acids 56-70 of the human tyrosinase polypeptide (abbreviated Tyrosinase₅₆₋₇₀) of Accession No. NP_000363.1 of the GENBANK® biosequence database. SEQ ID NO: 6 is also abbreviated herein as “AQN”.

### DETAILED DESCRIPTION

Despite the huge advancements in cancer therapies and treatments over the past decade, most patients with metastasized melanoma still die from the disease. This poor prognosis largely results from resistance to conventional chemotherapies and other cytotoxic drugs. Previously identified were six (6) antigenic peptides derived from melanomas that proved efficacious for activating CD4⁺ T cells in clinical trials for melanoma. To improve pharmacodynamics, pharmacokinetic and toxicological parameters, each of the six melanoma helper peptides were individually encapsulated within their own nanoliposomes. These liposomes were modified as described herein to account for differences in the peptides' chemical properties, resulting in three distinct formulations. To further enhance immunogenicity, KDO2, a TLR4 agonist, was also incorporated into the lipid bilayer of all nanoliposome formulations. Disclosed herein is that these liposomes, loaded with the different melanoma helper peptides, can be readily mixed together and simultaneously delivered without toxicity in vivo. These liposomes are capable of being diffused to the secondary lymphoid organs very quickly and for at least 6 days. In addition, it has been shown that these immunogenic liposomes enhanced immune responses to specific peptides ex vivo. Lipid-based delivery systems, including nanoliposomes and lipid nanoparticles, have now been validated for pharmacological (small molecules, bioactive lipids) and molecular (mRNA, siRNA) therapeutic approaches. However, the utility of these formulations as cancer vaccines, delivering antigenic peptides, has not yet achieved the same degree of commercial success. Described herein is the novel and successful development of a nanoliposome-based cancer vaccine for melanoma. These vaccines help to circumvent drug resistance by increasing a patient's T cell response, making them more susceptible to check point blockade therapy.

### I. DEFINITIONS

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a” “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table:

Amino Acid Codes and Functionally Equivalent Codons

| | 3- | 1- | |
|---|---|---|---|
| | Letter | Letter | |
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is me\ant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the compositions of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the compositions of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

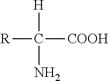

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table:

Exemplary Conservative Amino Acid Substitutions

| Group | Characteristics | Amino Acids |
|---|---|---|
| A. | Small aliphatic, nonpolar, or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. EXEMPLARY EMBODIMENTS

In some embodiments, the presently disclosed subject matter relates to compositions comprising stable KDO2-containing nanoliposomes. In some embodiments, the KDO2-containing nanoliposomes comprises a lipid component comprising, consisting essentially of, of consisting of DSPC, DOPE, PEG(2000)-PE, KDO2, Cholesterol, Rhodamine or DiD, and optionally DOTAP and/or DHP. The particular lipid compositions of the KDO2-containing nanoliposomes disclosed herein can be modified as desired, as can the buffers in which the KDO2-containing nanoliposomes are provided, such that the properties of the KDO2-containing nanoliposomes including but not limited to their abilities to encapsulate cargoes such as but not limited to immunogenic or other biologically active peptides are optimized.

As such, in some embodiments the KDO2-containing nanoliposomes are neutral liposomes. In some embodiments, the neutral liposomes comprise a lipid component comprising, consisting essentially of, of consisting of DSPC, DOPE, PEG(2000)-PE, KDO2, Cholesterol, and Rhodamine or DiD at a molar ratio of about 4.6:1.14:0.25:0.01:3.00:0.02, optionally in a buffer comprising about 1.33 mg/ml sodium bicarbonate in a 1:2 solution of Lactated Ringers solution (LR) and water. In some embodiments, the KDO2-containing nanoliposomes are cationic liposomes. In some embodiments, the cationic liposomes of the presently disclosed subject matter comprise a lipid component comprising, consisting essentially of, of consisting of DSPC, DOPE, PEG(2000)-PE, KDO2, Cholesterol, Rhodamine or DiD, and DOTAP at a molar ratio of about 4.12:1.90:0.25:0.01:3.00:0.02:0.70, optionally in a buffer comprising about 5 mg/ml sodium bicarbonate in water. In some embodiments, the KDO2-containing nanoliposomes are anionic liposomes. In some embodiments, the presently disclosed anionic liposomes comprise a lipid component comprising, consisting essentially of, of consisting of DSPC, DOPE, PEG(2000)-PE, KDO2, Cholesterol, Rhodamine or DiD, and DHP at a molar ratio of about 3.91:1.81:0.25:0.01:3.00:0.02:1.00, optionally in a buffer comprising about 1 ml/ml sodium bicarbonate in water or a buffer comprising a 1:9 ratio of 2-(N-morpholino) methanesulfonic acid (MES) buffer and water.

In some embodiments, the KDO2-containing nanoliposomes encapsulate one or more biologically active agents. Any biologically active agent for which delivery to a subject, or a cell, tissue, organ, or other target site in a subject, is desired can be encapsulated by a KDO2-containing nanoliposome of the presently disclosed subject matter.

In some embodiments of the presently disclosed subject matter, the KDO2-containing nanoliposomes encapsulate one or more peptides, which in some embodiments can be immunogenic peptides. In some embodiments, the one or more immunogenic peptides are peptides associated with malignant melanoma. In some embodiments, the peptides associated with malignant melanoma comprise, consist essentially of, or consist of amino acid sequences that are subsequences of a protein selected from the group consisting of tyrosinase, gp100, MAGE-1,2,3,6, Melan-A/MART-1, and MAGE-3. In some embodiments, the tyrosinase peptide comprises, consists essentially of, or consists of amino acids 56-70 and/or 386-406 of human tyrosinase; and/or the gp100 peptide comprises, consists essentially of, or consists of amino acids 44-59 of human gp100; and/or the MAGE-1,2,3,6 peptide comprises, consists essentially of, or consists of amino acids 121-134 of human MAGE-1,2,3,6; and/or the Melan-A/MART-1 peptide comprises, consists essentially of, or consists of amino acids 51-73 of human Melan-A/MART-1; and/or the MAGE-3 peptide comprises, consists essentially of, or consists of amino acids 281-295 of human MAGE-3. In some embodiments, the compositions comprise 1, 2, 3, 4, 5, or 6 different peptides selected from the group consisting of tyrosinase peptides, gp100 peptides, MAGE-1,2,3,6 peptides, Melan-A/MART-1 peptides, MAGE-3 peptides, and combinations thereof.

### Pharmaceutical Compositions and Administration

The presently disclosed subject matter is also directed to methods of administering the compounds of the presently disclosed subject matter to a subject. Thus, in some embodiments of the presently disclosed subject matter, the compositions are pharmaceutical compositions, optionally pharmaceutical compositions that are pharmaceutically acceptable for use in mammals such as but not limited to humans.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition subcutaneously, intraperitoneally, into adipose tissue, and/or intramuscularly into the subject.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isostearyl alcohols, polyoxyethylene sorbitol and sorbitan esters, microcrystalline cellulose, aluminum metahydroxide, bentonite, kaolin, agar-agar and tragacanth, or mixtures of these substances, and the like.

In some embodiments wherein a composition of the presently disclosed subject matter is desired to induce an immune response, the compositions of the presently disclosed subject matter can further comprise an adjuvant. In some embodiments, the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

The presently disclosed subject matter also related to methods for using the compositions of the presently disclosed subject matter for various purposes. For example, in some embodiments the presently disclosed subject matter also relates to methods for treating and/or preventing malignant melanoma. In some embodiments, the methods comprise, consist essentially of, or consist of administering to a subject in need thereof an effective amount of a composition as disclosed herein. In some embodiments, the subject in need thereof is a human.

Additionally, in some embodiments the presently disclosed subject matter relates to methods for inducing anti-melanoma immune responses in subjects. In some embodiments, the methods comprise, consist essentially of, or consist of administering an effective amount of a composition as disclosed herein to a subject in need thereof. In some embodiments, the subject in need thereof is a human.

Thus, in some embodiments the presently disclosed subject matter relates to uses of the presently disclosed compositions, including but not limited to compositions comprising, consisting essentially of, or consisting of stable KDO2-containing nanoliposomes encapsulating one or more tyrosinase peptides, gp100 peptides, MAGE-1,2,3,6 peptides, Melan-A/MART-1 peptides, MAGE-3 peptides, or any combination thereof for treating and/or preventing malignant melanoma and/or for inducing an anti-melanoma immune response in subjects.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Encapsulation of Melanoma Peptides within Nanoliposomes. The six melanoma peptides that induce melanoma-reactive CD4⁺ T cells in patients as well as their protein epitope and ideal pH range for solubility are provided in Table 1. A neutral formulation was initially chosen to encapsulate all peptides and then, through an iterative process, modified formulations to optimize encapsulation efficiencies for several of the peptides. Through this process, three different nanoliposome formulations (neutral, cationic, and anionic) suitable for distinct peptides were identified. However, all of the presently disclosed formulations contained the same base lipid components, including 1,2-distearoyl-sn-glycero-3-phosphocholine (DSPC); 1,2-Dioleoyl-sn-glycero-3-phosphoethanolamine (DOPE); 1,2-distearoyl-sn-glycero-3-phosphoethanolamine-N-[methoxy(polyethylene glycol)-2000] (ammonium salt) (PEG2000PE); KDO2; cholesterol; and a fluorophore (either rhodamine or 1,1′-Dioctadecyl-3,3,3′,3′-Tetramethylindodicarbocyanine, 4-Chlorobenzenesulfonate Salt (DiD)). Each formulation contained DSPC and DOPE at a 2.14:1 molar ratio to form a stable, spherical nanoparticle. In addition, cholesterol at a 30 molar percent was incorporated to increase rigidity and to reduce leakiness. PEG(2000)-PE at 2.5 molar percent was incorporated for biological stability, KDO2 was added to all formulations at 0.1 molar percent, and a fluorescent probe (rhodamine or DiD) was added at 0.2 molar percent for subsequent biodistribution imaging studies. Keeping the molar ratios of these key components constant, dihexadecyl phosphate (DHP) was incorporated at 10 molar percent for the anionic formulation and positively charged lipid 1,2-dioleoyl-3-trimethylammonium-propane (chloride salt) (DOTAP) at 7 molar percent for the cationic formulation. All lipid components were dissolved in chloroform and mixed in the ratios shown in Table 2. The lipid mixtures were then dried down in a nitrogen blower for roughly 2 hours until all of the chloroform was evaporated. Then, the peptide solutions, (or 1×PBS for the “ghost” formulation), was added to the dried down lipids, the tubes were then vortexed and placed in a heat shaker at 60° C. for 2 hours, followed by sonication in a 60° C. sonic bath for roughly five minutes. After sonication, the liposomes were extruded through a 100-nanometer pore membrane eleven times to create uniformly sized liposomes. The extruded liposomes were subsequently run through a Sepharose gel bead column to separate the liposomal drugs from the free drug. The solution was analyzed using Dynamic Light Scattering (DLS; polydispersity and average hydrodynamic diameter of particles in solution), and mass spectrometry.

To further ensure optimal encapsulation efficiency of the MHPs, distinct dissolution buffers tailored to each peptide's optimal pH range and previously optimized for in vivo injection of the free peptides into patients enrolled in earlier clinical studies were utilized. Specifically, for the neutral peptide formulations, 1.33 mg/ml sodium bicarbonate (NaHCO₃) in a 1:2 solution of lactated Ringers solution (LR) and water respectively (B1); for the cationic formulation, 5 mg/ml NaHCO₃ in water (B2); for the WNR anionic formulation, 1 mg/ml NaHCO₃ in water (B3); and for the TSYVKVLHHMVKISG (TSY; SEQ ID NO: 1) anionic formulation, 1:9 ratio of 2-(N-morpholino) methanesulfonic acid (MES) buffer and water (B4).

Liposomal stability and peptide release studies were conducted by storing the liposomes in different conditions and media. The liposomes were monitored over a 5-week period in both 1×PBS and 10% fetal bovine serum, under refrigerated, room temperature, and body temperature storage conditions. DLS studies, as well as centrifugation followed by mass spectrometry of the supernatant and reconstituted liposome solutions were run to monitor liposome stability and peptide release, respectively.

In vivo murine studies. A 6-day study was performed to determine the biodistribution of the fluorescent nanoliposomes, utilizing daily live-mouse imaging with an in vivo Imaging System (IVIS). For this study, four mice received a DiD fluorescently labeled 6MHP-KDO2-nanoliposome injection. Two mice were injected subcutaneously (SQ) into either flank and two mice were injected intravenously (IV). Each peptide nanoliposome had been previously formulated and stored separately. On the day of the experiment, all six formulations were mixed together in ratios that contained 0.4 μg of each peptide. The mice were administered isoflurane anesthesia. On day zero, once asleep, the mice were injected with the 6MHP-KDO2-nanoliposome mixture and then transferred immediately to the IVIS instrument. Imaging was conducted within 30 seconds (or less) of injection. For IVIS® Spectrum Image (Caliper Life Sciences, Hopkinton, Massachusetts, United States of America) collection and processing, tomographic fluorescent images were collected of all mice using epi-illumination on a IVIS® Spectrum scanner following anesthetization with inhaled isoflurane inside a conduction chamber. Live imaging was then performed daily for six days. On the sixth day, the mice were euthanized, and their organs were harvested for follow-up ex vivo studies. The organs selected for harvest included the lungs, liver, spleen, kidneys, and the lymph nodes. Organs from the in vivo study were transferred to 24-well plates, and fluorescent imaging data were collected on the IVIS® Spectrum Image system for qualitative determination of biodistribution. Fluorescent image collection used LIVING IMAGE® software by Caliper Life Sciences. Image processing was done by importing LIVING IMAGE® files into Aura imaging software by Spectral Instruments Imaging, and then performing region of interest (ROI) measurements to quantify the fluorescence emitted from the mice. The fluorescent light images were collected in the same manner for the harvested organs following the live animal imaging once the mice were sacrificed. The measurement for fluorescence is mean radiant efficiency within the drawn ROI. Radiant efficiency describes the fluorescent energy emitted from the specimen as a fraction of the excitation fluorescent radiation released by the scanner and incident upon the specimen. Mean radiant efficiency is unitless and is used so that comparisons may be made among mice and harvested organs of different sizes, and consequently slightly differing areas within ROIs. ROIs were drawn as subject ROIs according to the Living Imaging® software user manual (Caliper Life Sciences, 2012).

Ex Vivo Lymphocyte Stimulation using Immunogenic TSY encapsulated nanoliposomes. Freshly obtained (cryopreserved) peripheral blood mononuclear cells (PBMC) and lymphocytes from the sentinel immunized nodes (SIN) of two patient donors (under IRB protocols, clinical trial Mel41 (NCT00089219; IRB #10464; Slingluff et al., 2008b) were utilized to assess CD4⁺ T cell proliferation after treatment with the anionic TSY (MAGE-3₂₈₁₋₂₉₅ peptide; Table 1) MIP-KDO2 nanoliposome. Firstly, a cell viability study was performed using a live-dead marker and flow cytometry to ensure that cell viability was not impacted by the presence of either the peptides or the liposomes. In this study, SIN lymphocytes from the two patient donors (SIN1 and SIN2) were treated with a variety of free peptides and liposome combinations. For the free peptide group, the treatments included: no peptide and GAG peptide as negative controls; a mixture of all 6MHPs, and the single TSY peptide of interest. For the liposome group, the cells were treated with ghost liposomes; liposomes containing KDO2 but no peptide; and TSY containing liposomes both with and without KDO2. A control cell line was used as a viability comparison. Next, in order to assess CD4 T cell proliferation, the SIN1 and SIN2 cell lines were split into 5 treatment groups. These were: free non-encapsulated TSY; a ghost nanoliposome, a ghost KDO2 nanoliposome, a TSY encapsulated (no KDO2) nanoliposome, and a combinatorial KDO2/TSY nanoliposome (full vaccine). A CFSE dye-dilution proliferation assay was performed to evaluate the donor immune response by flow cytometry. Specimens were thawed and labeled with carboxyfluorescein diacetate (CFSE; VYBRANT® CFDA SE Cell Tracer Kit, INVITROGEN™, ThermoFisher Scientific, Waltham, Massachusetts, United States of America) according to the manufacturer's instruction, with a final dye concentration of 1 μM. Two hundred thousand labeled cells were added to flat bottom wells of a 96-well cluster plate (Falcon, ThermoFisher Scientific) containing peptides in solution or in nanoparticle form. Final peptide concentration was 2 μg/mL in a final culture volume of 0.2 mL. A media-only and an HIV GAG peptide were also utilized as negative controls (Benati et al., 2016). To avoid cell starvation from the high PBS content of the nanoliposomes' solvent relative to the necessary culture media nutrient content to sustain the culture during incubation, cells and liposome-containing formulations of peptide (and liposome controls) were first incubated (“pulsed”) for two hours at 37° C. followed by centrifugation to pellet the cells (and absorbed or internalized nanoparticles). The “pulsing” medium supernatant was removed and replaced with complete culture medium consisting of AIM V™ (Gibco/Life Technologies, ThermoFisher Scientific) supplemented with 5% human AB serum (Gemini Bio Products, West Sacramento, California, United States of America). Treatments were incubated for five days. At the end of incubation, cells were collected and labeled with a LIVE/DEAD™ fixable dye (Aqua; ThermoFisher Scientific) followed by labeling with CD3 v450, CD4 PE, and CD8 PE-Cy7 (BD Biosciences, Franklin Lakes, New Jersey, United States of America). Cells were acquired on a FACSCANTO™ II flow cytometer (BD Biosciences) maintained by the Carter Immunology Center at the University of Virginia and the data analyzed using FlowJo software (version 10; BD Biosciences).

### Example 1

### Physio-Chemical Characterization of 6MHP Nanoliposomal Formulations

Each of the six Melanoma Helper Peptides (6MHP) were initially incorporated in a neutral liposome formulation containing: (DSPC); (DOPE); (PEG2000PE); KDO2; cholesterol; and a fluorophore in ratios of 4.60:2.14:0.25:0.01:3.00:0.02 respectively (see Table 2). After quantifying initial encapsulation masses, an iterative approach was utilized to improve encapsulation via modifying the charge and lipid ratios of the liposomal formulation as well as the buffer for the peptide. The proportional improvements in encapsulation as well as the encapsulation efficiencies are depicted in Table 3.

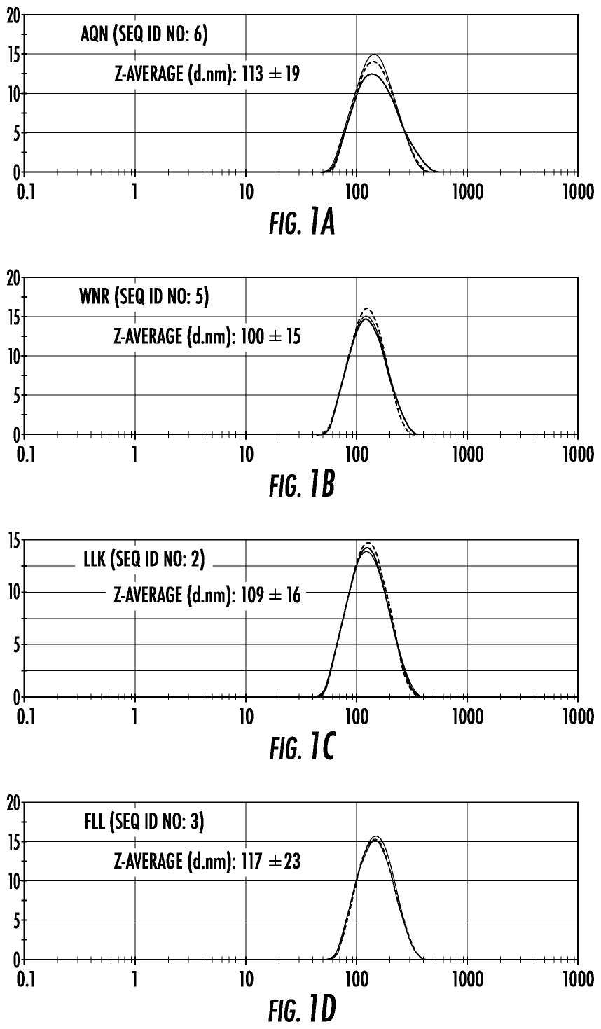

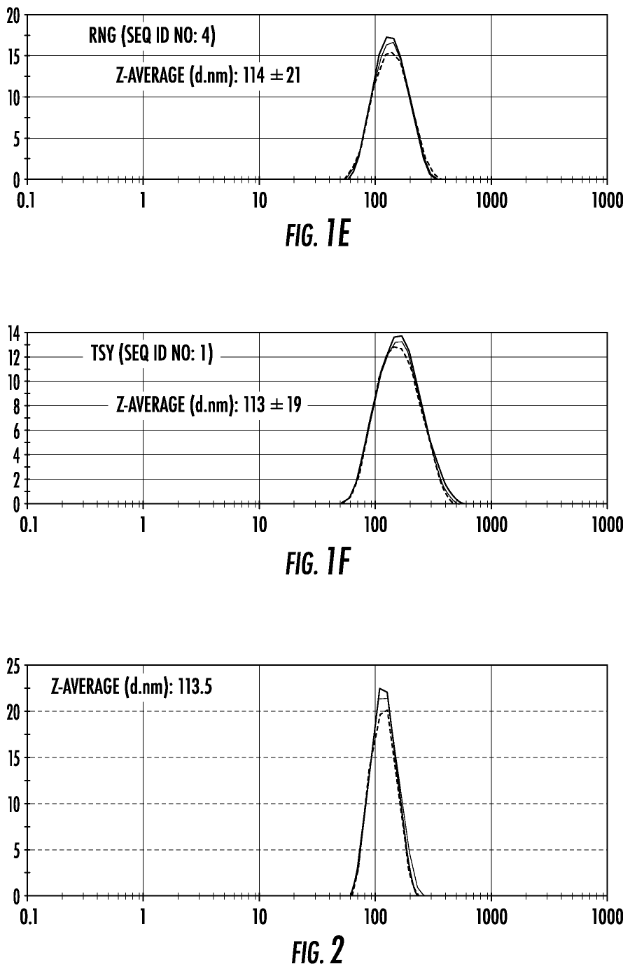

Using dynamic light scattering (DLS, Malvern Instruments), it was demonstrated that all 6 optimized formulations displayed stable, consistent and homogeneous size distribution, with an overall average size range of 113±8 nm when averaged among all six formulations (FIG. 1). Furthermore, FIG. 2 shows that all six formulations could be mixed together and still remain stable in suspension, providing confidence that the charged nanoliposomes were not interacting with each other to cause destabilization or aggregation. The mixed sample gave a Z-Average size of 113.5 nm, which is in agreement with the average size distribution of the individual nanoliposomes.

In addition, results of a 5-week long release kinetics study and analysis by LC-MS did not detect any measurable amount of peptides present in the MIP-KDO2-nanoliposome supernatant when stored at 4° C. These data suggested that the nanoliposomes did not have a significant release of encapsulated peptide from their core for at least that length of time, while under typical refrigerated storage conditions. Additionally, the supernatant collected remained clear, providing confidence that the fluorophore was not released from the liposome and DLS analyses still showed good liposome stability. Conversely, at body temperature, greater than 50% of the peptide concentration was released within the first 24 hours. DLS data showed that the liposome remained intact, even in 10% bovine serum and the supernatant remained clear of fluorophore. After one week, the liposomes kept at room and body temperature did appear to begin breaking down, shown by an increased number of peaks in DLS analyses.

### Example 2

### Murine Biodistribution Studies

A biodistribution study using a mixture of all 6 MHP formulations was performed using an IVIS® Spectrum imaging system. Since each liposome formulation contained a different peptide mass, the mixture was prepared to contain equal amounts of each peptide (i.e., different volumes of each liposome solution to maintain equal peptide delivery). For this particular study, the nanoliposome vaccine was prepared using (DiD) fluorophore, as DiD has a far-infra red emission spectrum conducive for live animal imaging via IVIS.

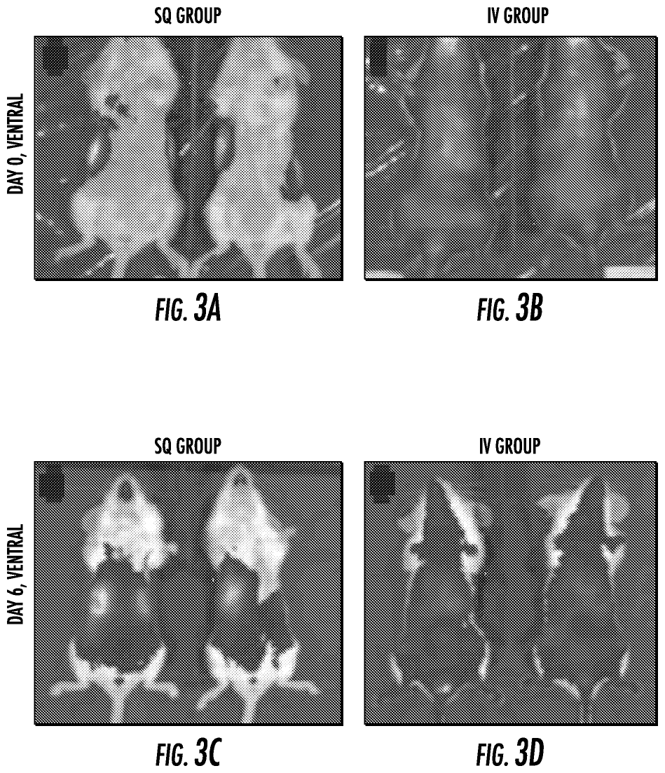

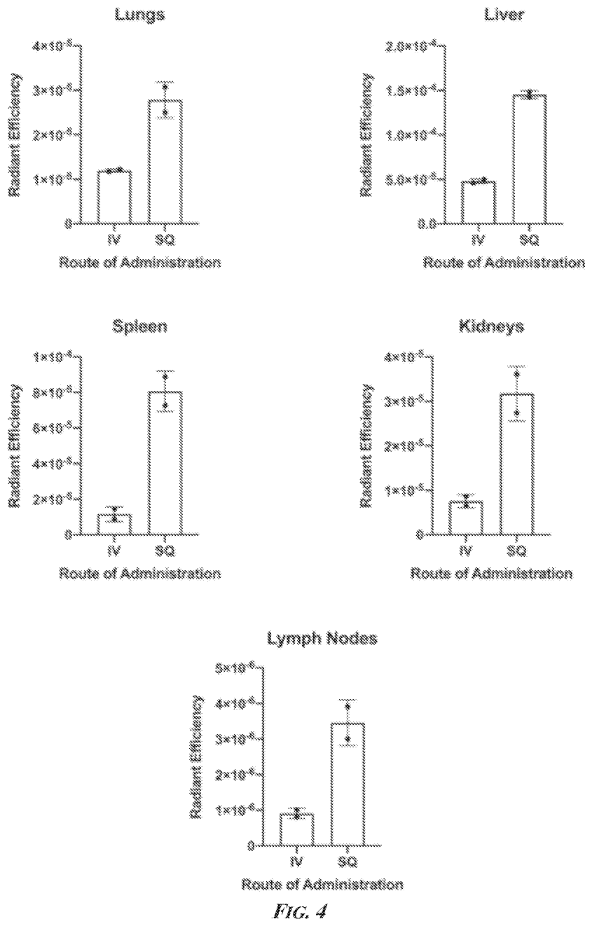

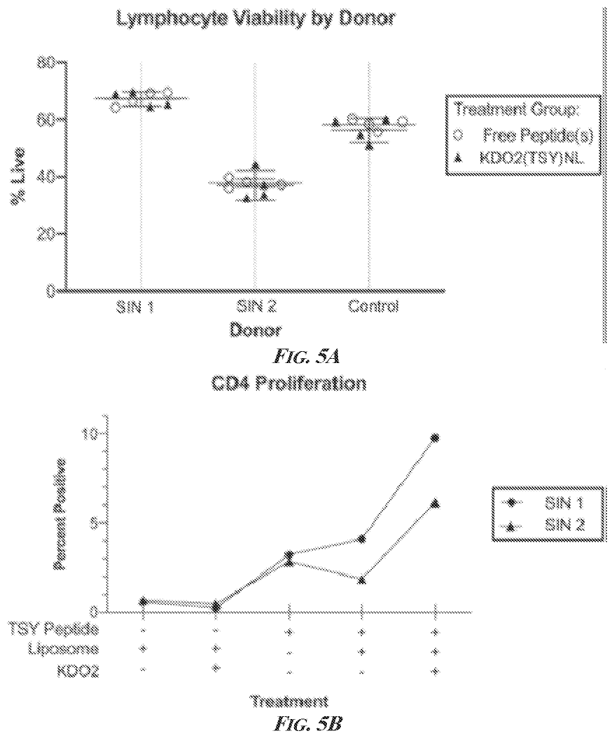

Routes of administration, SQ vs. IV, were compared. The IVIS images on day zero (immediately after injection) and day 6 (six days after injection) are shown in FIG. 3. The mice injected SQ displayed little bio-distribution on day zero (FIG. 3A), which intensified within a region consistent with liver and spleen at day 6 (FIG. 3C). In contrast, images of the mice injected IV with nanoliposome vaccines show rapid, systemic biodistribution in both mice on day zero (FIG. 3B), but the tissue fluorescence diminished by day 6 (FIG. 3D) as compared to the fluorescence on day zero. On day 6, the mice were sacrificed and their organs were harvested. Quantitative fluorescence measurements were performed using Aura region of interest (ROI) programming and software (see Materials and Methods above), and these results are shown in FIG. 4.

For all organs, the mice injected SQ displayed a higher concentration of fluorescent liposomes within the harvested organs than the IV-injected group. No overt signs of toxicity (lethargy, loss of appetite, weight loss) were observed in any of the mice over the study. Taken together, these data suggested that both IV and SQ administration of a KDO2-6MHP nanoliposome was safe, and that SQ administration of the KDO2-6MHP nanoliposome offered a robust, delivery of cargo to critical secondary lymphoid organs (spleen and lymph nodes) that persisted at least six days.

### Example 3

### Activation of CD4⁺ T Cells from Patients by a KDO2-MIP Nanoliposome

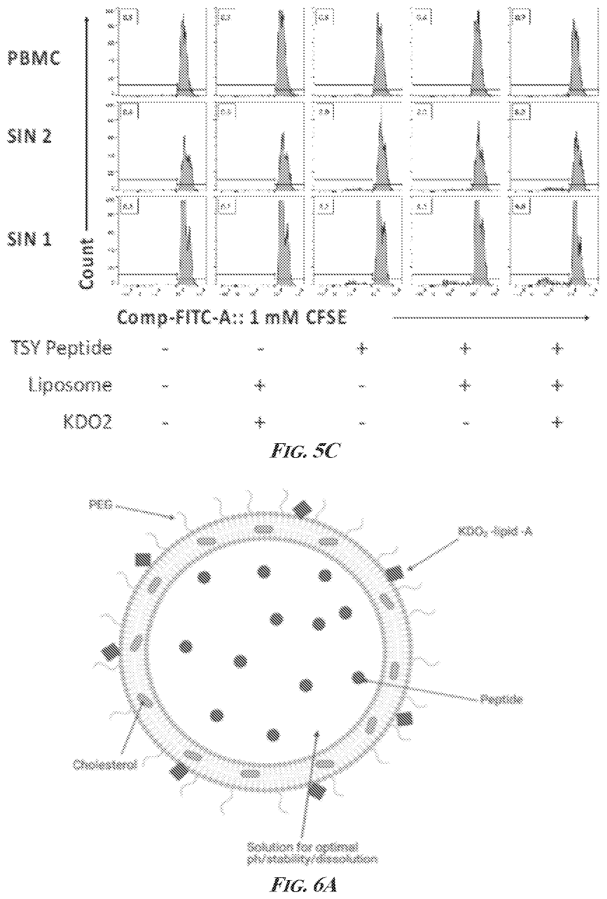

Using blood samples from melanoma patients who had previously been vaccinated with a 6MHP vaccine on the Mel41 trial and had developed a robust CD4⁺ T cell immune response to the TSY antigen (Slingluff et al., 2008b), lymphocyte viability and CD4⁺ T cell proliferation to the TSY nano-formulations ex vivo were analyzed. Under IRB guidance, fresh lymphocytes and sentinel immunized nodes (SIN) had been previously collected from these patients during that prior clinical trial. SIN were nodes draining the vaccine site, identified by lymphatic mapping techniques (Slingluff et al., 2008a; Slingluff et al., 2008b). Using a live-dead marker and flow cytometry, no discernable differences in cell viability between each of the patient samples re-treated with free non-encapsulated TSY peptide and the nanoliposomal KDO2/TSY formulation were observed; however, the SIN sample from patient 2 (SIN2) had a decreased viability compared to SIN patient 1 (SIN1; see FIG. 5A). Viability was equivalent between a control group of patients samples that were not previously immunized with peptides (FIG. 5A). Since there was no apparent toxicity to incubation with nanoliposomes, aliquots of each cell preparation were treated with each of the following: (1) an empty ghost liposome; (2) a liposome containing KDO2 but no peptide; (3) Free TSY peptide (no liposome); (4) TSY encapsulated within a liposome; and (5) TSY within an immunogenic (KDO2 containing) liposome. Then, the average proliferation of CD4⁺ gated cell populations for various culture treatment conditions was assessed (FIG. 5B) by CFSE dilution over 5 days. Cell cultures were expanded from the harvested SIN biopsy of each donor, and CD4⁺ T cell proliferation was assessed by CFSE dye dilution. Proliferation is reported as the percentage of CD3⁺ CD4⁺ gated population that were dividing (CFSE-diluted). Compared to all controls, both SIN1 and SIN2 donor cells responded to the combinational therapy. FIG. 5C shows the 2D histograms of the same data against a negative control. Peripheral blood samples from each patient had no significant response to any of the treatments. While an expected CD4⁺ T cell proliferation response was observed with free TSY and liposomal TSY, the presently disclosed data suggested an advantage of combining the peptide vaccine with a TLR4 agonist. It is worth noting that neither the liposome alone nor the KDO2-containing liposome alone triggered CD4⁺ proliferation, and thus KDO2 only acted to enhance the response to the peptide of interest. It should also be noted that while the SIN2 patient response was weaker than SIN1 (FIG. 5B), this could be attributed to the slightly lower viability (FIG. 5A) (and hence fewer live cells present) in the SIN2 donor cell population. Taken together, an immunogenic response to the KDO2/MHP nanoliposome ex vivo was demonstrated.

### Discussion of the Examples

As disclosed herein, nanoliposome formulations were developed to encapsulate a specific panel of six (6) melanoma helper peptides as a first-step toward clinical application as a new melanoma vaccine strategy. The six peptides varied in length, hydrophobicity/hydrophilicity, and isoelectric point, which required creating three different nanoliposomal formulations to encompass them. These six peptides had previously shown modest immunogenicity and clinical activity as a vaccine when injected in their free form with other immunogenic adjuvants. A goal was thus to improve future clinical efficacy through nano-enhancement strategies via simultaneous delivery of all 6MHPs and corresponding immunogenic adjuvants in a single nanoliposomal solution. In these investigations, KDO2, a TLR4 agonist, was incorporated into the lipid bilayer as an immune-stimulating adjuvant. The presently disclosed results demonstrated that the individual liposomal formulations remained stable after being mixed together and that they could be simultaneously delivered without apparent toxicity in vivo. It was also shown that via SQ injection, these 6MHP-loaded liposomes were capable of diffusing rapidly to secondary lymphoid organs and appeared to remain in circulation for at least 6 days. It was further shown that these immunogenic liposome formulations significantly enhanced immune responses to specific peptides ex vivo.

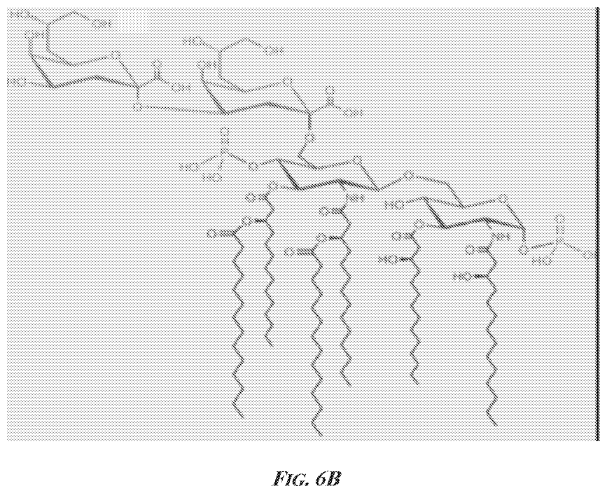

The initial development of the presently disclosed nano-enhanced vaccine was complicated, as it was necessary to encapsulate and ultimately deliver six different peptides simultaneously. A nanoliposome framework was selected because of its versatility. Nanoliposomes can be made with a variety of different lipids to construct the lipid bilayer. They can be made to be neutral or charged, and the exterior surface can be modified chemically for the addition of targeting ligands that enhance liposome delivery; and/or stealth properties that increase biocompatibility and circulation time in the body (Tassa et al., 2010; Cisterna et al., 2016; Tiet & Berlin, 2017). Hydrophilic compounds are typically encapsulated within a nanoliposome's aqueous interior, while hydrophobic (or lipophilic) compounds typically embed within the lipid bilayer. However, development and optimization of nanoliposomes is not always a straightforward process, as overall charge, distribution of charge, and size, as well as ionic buffer strength affect encapsulation efficiency. Each of the presently disclosed peptides had different physical properties, so a single liposome formulation was not optimal for this range of peptides. Instead, three formulations were engineered and each peptide encapsulated based upon pH-dependent solubility. While each formulation may have had a different surface charge or be formulated with a different buffer system, they still all included a fixed PC/PE ratio that maintained stability, a fixed cholesterol content that prevented leakiness, a fluoroprobe to facilitate in vivo imaging, a reduced PEG brush to help trigger the body's T cell response to these liposomes, and a low concentration of the TLR4-agonist, KDO2, to enhance the adaptive immune response without inducing systemic toxicities. FIG. 6A shows a schematic diagram of an exemplary base nanoliposome formulation of the presently disclosed subject matter, and FIG. 6B shows the chemical structure of KDO2-Lipid A.

The new formulations and pH-controlled buffers improved the encapsulation efficiency of most of the peptides compared to the use of a generic neutral nanoliposome formulation. TSY was improved the most. TSY was most stable in pH 5-5.5; thus, PBS did not provide the ideal buffer conditions, while the MES buffer used in the presently disclosed optimized formulations was much more favorable. The anionic liposome formulation also aided in improving the encapsulation of the positively-charged TSY peptide. Similarly, AQN, which had very poor encapsulation in the presently disclosed neutral formulation, also achieved huge encapsulation enhancements when dissolved in a buffer that maintained an alkaline pH and by using a cationic charged nanoliposome to further enhance encapsulation of the negatively charged peptide. By contrast, encapsulation of WNR, which has a slightly positive charge, was only marginally improved after switching to an anionic liposome and buffer that maintained a pH of 7.0-8.0. LLK, FLL, and RNG achieved reasonable encapsulation values in the presently disclosed neutral formulation, so significant improvements with a switch of buffers was not expected. However, for LLK and FLL, but not RNG, dissolving the peptides in LR/NaHCO₃ buffer instead of PBS slightly improved encapsulation efficiencies. Based on previous clinical studies, where 200 μg of each peptide was delivered to human patients (Slingluff et al., 2008b), it was estimated that a liposomal loading value of ˜50 μg/ml should yield an effective immunologic dose. The presently disclosed studies showed that LLK, FLL, and TSY could be reengineered to reach and/or exceed this therapeutic dose. For AQN, WNR, and RNG, pH-dependent active methods of liposomal loading can also be designed to further enhance encapsulation of these peptides within these nanoformulations. However, based upon previous studies from the instant co-inventors (Barth et al., 2019) it might be expected that the nanoformulations in fact better protect and deliver the peptides, allowing suboptimal encapsulation efficiencies to now reach target therapeutic doses.

To further improve liposomal MHP encapsulation, the peptides are modified via techniques such as myristoylation (Perez Socas & Ambroggio, 2020) or palmitoylation (Stolk et al., 2020). Both of these methods have shown to enhance peptide-lipid interactions, allowing for intercalation of the peptides within the lipid bilayer. This process was not chosen initially for several reasons. First, as cholesterol, KDO2, and the presently disclosed fluorophore were already intercalated within the lipid bilayer, it was desired that the peptides sit within the aqueous core of the liposomes in their native forms. There were also concerns that incorporation within the lipid bilayer might lower the efficacy of the presently disclosed therapy due to prolonged release kinetics and/or destructive interaction/competition with the other adjuvants already in the lipid bilayer. It was also unclear about how such modifications (and changes in peptide configuration) might affect the immunogenicity of the parent MHP. As part of this ongoing work, whether these and similar modifications preserve or alter the immunogenicity of the 6MHPs is also tested.

A major innovation of the presently disclosed nano-encapsulated approach is the incorporation of the immunogenic KDO2-lipid A into the presently disclosed core nanoliposome formulation. The present study demonstrated the feasibility of creating customized nanoliposomes, containing varying peptides, which could then be mixed for storage and co-administration. These data provided a platform on which to build even more promising nanoliposome strategies. In some embodiments, an antibody that can target the adjuvanted nanoliposomes specifically to dendritic cells is also added. In murine studies, very strong circulating T cell responses, representing about 50% of circulating T cells, have been induced by IV vaccination with peptides plus a TLR agonist and a CD40 antibody that targets antigen-presenting cells (TriVax; Cho & Celis, 2009; Barrios & Celis, 2012; Hu et al., 2015; Eggermont et al., 2016) However, comparable systemic dosing of those agents for humans is not likely achievable without unacceptable toxicity. If, on the other hand, these three agents could be co-administered in a nanoparticle targeted to dendritic cells (DC), it could prove as effective with a much lower dose. Other versions of the presently disclosed immunogenic liposomes can include bioconjugation strategies (EDC/NHS) for binding of CD40 Ab to the surface of the liposomes.

The presently disclosed biodistribution studies, conducted in healthy murine subjects, suggested that SQ administration could be a preferred method of delivery for the presently disclosed nano-vaccines. Systemic (IV) delivery seems to rapidly distribute the liposomes non-specifically throughout the body immediately after injection, and also appears to then produce a more rapid clearing of them from the body. On the other hand, SQ delivery allows for a slower and more controlled distribution. The extent to which the fluorescent tracer may be released over time in vivo remains unclear. However, as the fluorophore is conjugated to a lipid within the bilayer, significant amounts of tracer to be released is not expected until the liposomes themselves begin breaking down. The presently disclosed benchtop studies at body temperature in 10% bovine serum showed, that while peptide release after 24 hours was observed, the liposomes themselves remained stable for several days. There is reason to be confident that the fluorescent distributions imaged immediately after injection on day zero (FIGS. 3A and 3C) are representative of liposome distribution at that time. Unlike IV delivery, SQ delivery does not immediately distribute systemically but does show rapid appearance of the liposomes within the lymph nodes and spleen. This was encouraging and implied that the nanoliposomes exhibited immunogenicity. It also suggested that even without specific dendritic cell targeting, the nanoliposomes distributed in tissues with high concentrations of dendritic cells, which would be expected to support T cell activation. More detailed benchtop tests, to specifically monitor fluoroprobe release over time, as well as increased frequency of live animal imaging are also conducted. This work is further extended into a B16 melanoma mouse model. This requires exchanging the presently disclosed humanized antigens with mouse antigens, which could require some alterations to the nanoformulations. However, this kind of study permits evaluation of biodistribution, pharmacokinetics (PK), and immunogenicity of the KDO2-nanoliposomes; as well as determination of pharmacodynamics (PD) for efficacy of the presently disclosed nanoformulations in a mouse model. Additionally, these mouse studies can be repeated with depletion of dendritic cells to confirm the reliance on those cells.

Summarily, a goal of the present work was to test whether the 6MHPs could be encapsulated into nanoliposomes with a TLR agonist and whether these would be stable and show preliminary evidence of enhanced immunogenicity. In preliminary investigations, the TSY peptide consistently achieved the highest liposomal encapsulation values and was also strongly immunogenic in a melanoma patient population (Slingluff et al., 2008a). The present co-inventors also had access to two patient samples, previously documented to exhibit a strong TSY immunogenic response. Thus, as a proof-of concept, proliferative responses of human CD4⁺ T cells to the TSY peptide ex vivo were evaluated using these two patient samples. The presently disclosed assays showed an enhanced immunogenic response with the peptide encapsulated within a KDO2-nanoliposome, compared to formulations without KDO2 and compared to free peptide. It was expected that this enhancement was mediated by dendritic cell activation and subsequent helper peptide presentations to melanoma-reactive T cells. These results are in line with clinical data demonstrating that simultaneous delivery of a TLR4 agonist with immunogenic peptides enhances T-cell activation (Melssen et al., 2019). These initial studies provide a basis for carrying forward this study, first by performing ex vivo activation of the five remaining antigens individually, as well as ex vivo analysis of the “full vaccine” with a mixture of all six peptide nanoliposomes.

The presently disclosed results show promise in the use of custom-designed immunogenic (KDO2) nanoliposomes as the delivery vehicle for cancer vaccines. Co-delivery of antigens plus the TLR agonist KDO2 to antigen-presenting cells offers promise to enhance immune responses to melanoma antigens. Prior vaccines in humans commonly induce weak or transient T cell responses: by enhancing those immune responses, this new strategy offers promise to overcome weak antitumor immunity and to enable immune-mediated control of melanoma. Moreover, by improving the delivery and efficacy of nano-cancer vaccines, drug resistance to current chemotherapies can also be overcome. The presently disclosed mouse studies show that a subcutaneous injection may have advantages over IV injection, as it allows the nanoliposome vaccine to concentrate and to persist within tissues and organs with high DC populations. This will provide an easier route to immunization that is cost effective and less invasive for the patients receiving care. Co-administering diverse adjuvants within a nanoliposome is expected to show even further enhanced responses in vivo, as it provides a way of targeted delivery, ensuring that all adjuvants are delivered directly and simultaneously to DCs.

TABLE 1
Amino Acid Sequences of the Six Melanoma Helper Peptides
Employed Herein*

| Epitope | | | Ideal |
|---|---|---|---|
| (protein, residues) | Amino acid sequences | Abbrev | pH range |
| MAGE-3₂₈₁₋₂₉₅ | TSYVKVLHHMVKISG | TSY | 5.0-5.5 |
| | (SEQ ID NO: 1) | | |
| MAGE-1, 2, 3, 6₁₂₁₋₁₃₄ | LLKYRAREPVTKAE | LLK | 6.0-8.0 |
| | (SEQ ID NO: 2) | | |
| Tyrosinase₃₈₆₋₄₀₆ | FLLHHAFVDSIFEQWLQRHRP | FLL | 6.0-8.0 |
| | (SEQ ID NO: 3) | | |
| Melan-A/MART-1₅₁₋₇₃ | RNGYRALMDKSLHVGTQCALTRR | RNG | 6.0-8.0 |
| | (SEQ ID NO: 4) | | |
| gp100₄₄₋₅₉ | WNRQLYPEWTEAQRLD | WNR | 7.0-8.0 |
| | (SEQ ID NO: 5) | | |
| Tyrosinase₅₆₋₇₀ | AQNILLSNAPLGPQFP | AQN | 8.5-9.0 |
| | (SEQ ID NO: 6) | | |

*Amino acid sequences correspond to human polypeptide sequences, with numbering in the first column corresponding to the amino acid positions in the noted polypeptide of the epitope recognized by the T cell receptor, which defines the specificity of the response. Abbrev: abbreviation used throughout the application. The ideal pH range required for stability and dissolution for each peptide is also provided.

TABLE 2
Finalized Liposome Formulations for the Neutral,
Cationic, and Anionic Nanoliposomes

| | Molar Ratio | | |
|---|---|---|---|
| | Neutral | Cationic | Anionic |
| Lipid Component | (FFL, LLK, RNG) | (AQN) | (WNR, TSY) |
| DSPC | 4.60 | 4.12 | 3.91 |
| DOPE | 2.14 | 1.90 | 1.81 |
| PEG(2000)-PE | 0.25 | 0.25 | 0.25 |
| KDO2 | 0.01 | 0.01 | 0.01 |
| Cholesterol | 3.00 | 3.00 | 3.00 |
| Rhodamine (or DiD) | 0.02 | 0.02 | 0.02 |
| DOTAP | — | 0.70 | — |
| DHP | — | — | 1.00 |
| Buffer | B1 | B2 | B3 (WNR) |
| | | | B4 (TSY) |

Formulations were developed to encapsulate different peptides based on their residual charge. A dissolution buffer maintaining each peptide's optimal pH range was also used during fabrication, where B1=1.33 mg/ml sodium bicarbonate (NaHCO₃) in a 1:2 solution of Lactated Ringers solution (LR) and water respectively; B2=5 mg/ml NaHCO₃ in water; B3=1 mg/ml NaHCO₃ in water; and B4=1:9 ratio of 2-(N-morpholino) methanesulfonic acid (MES) buffer and water.

TABLE 3
Initial Peptide Encapsulation in Nanoliposome Formulations Dissolved in 1X PBS

| | | | | Optimized | | |
|---|---|---|---|---|---|---|
| | Original mass | New | | mass | | Encap. |
| | encapsulation* | liposome | pH | encapsulation | Encap. | Efficiency |
| Peptide | (μg/ml) | formulation** | range | (μg/ml) | improvement | (%) |
| AQN | 0.02 ± 0.005 | Cationic (B2) | 8.5-9.0 | 6.25 ± 1.13 | 312X | 1.25 |
| WNR | 4.81 ± 0.09 | Anionic (B3) | 7.0-8.0 | 7.64 ± 0.99 | 1.59X | 1.53 |
| LLK | 34.56 ± 0.69 | Neutral (B1) | 6.0-8.0 | 57.26 ± 14.39 | 1.66X | 11.45 |
| FLL | 28.15 ± 2.05 | Neutral (B1) | 6.0-8.0 | 58.61 ± 4.96 | 2.08X | 11.72 |
| RNG | 22.37 ± 0.30 | Neutral (B1) | 6.0-8.0 | 19.18 ± 6.36 | 0.85X | 3.29 |
| TSY | 0.17 ± 0.001 | Anionic (B4) | 5.0-5.5 | 141.35 ± 2.18 | 831X | 28.27 |

*using neutral formulation and 1X PBS;
**Charge and buffer. The Table also shows the encapsulation (Encap.) efficiencies within the presently disclosed optimized nanoliposome formulations with each peptide dissolved in aqueous solutions that also maintain optimal pH and peptide stability and dissolution. Encapsulation efficiency is based upon initial 500 μg/ml peptide concentration. All peptides were calculated from a full vaccine, which included all lipid components (i.e., adjuvants, PEG, peptides). Each optimized mass encapsulation is based on n = 3 separate experiments, repeated in triplicate.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Barrios & Celis (2012) TriVax-HPV: An Improved Peptide-Based Therapeutic Vaccination Strategy against Human Papillomavirus-Induced Cancers. Cancer Immunol Immunother 61(8):1307-1317.
Barth et al. (2019) Sphingolipid Metabolism Determines the Therapeutic Efficacy of Nanoliposomal Ceramide in Acute Myeloid Leukemia. Blood Adv 3(17):2598-2603.
Benati et al. (2016) Public T Cell Receptors Confer High-Avidity CD4 Responses to HIV Controllers. J Clin Invest 126(6):2093-2108.
Bird et al. (1988) Single-chain antigen-binding proteins. Science 242:423-426
Blass & Ott (2021) Advances in the Development of Personalized Neoantigen-Based Therapeutic Cancer Vaccines. Nat Rev Clin Oncol 18(4):215-229.
Chiang et al. (2011) Adjuvants for Enhancing the Immunogenicity of Whole Tumor Cell Vaccines. Int Rev Immunol 30(2-3):150-182.
Cho & Celis (2009) Optimized Peptide Vaccines Eliciting Extensive CD8 T-Cell Responses with Therapeutic Antitumor Effects. Cancer Res 69(23):9012-9019.
Chung et al. (2020) COVID-19 Vaccine Frontrunners and Their Nanotechnology Design. ACS Nano 14(10):12522-12537.
Cisterna et al. (2016) Targeted Nanoparticles for Colorectal Cancer. Nanomed 11(18):2443-2456.
Dance (2017) Cancer Immunotherapy Comes of Age. Science 355(6330):1220-1222.
Devereux et al. (1984) A comprehensive set of sequence analysis programs for the VAX. Nucl Acids Res 12:387.
Eggermont et al. (2016) Prolonged Survival in Stage III Melanoma with Ipilimumab Adjuvant Therapy. N Engl J Med 375(19):1845-1855.
Fan & Moon (2015) Nanoparticle Drug Delivery Systems Designed to Improve Cancer Vaccines and Immunotherapy. Vaccines 3(3):662-685.
Fidler (1988) Targeting of Immunomodulators to Mononuclear Phagocytes for Therapy of Cancer. Adv Drug Delivery Rev 2(1):69-106.
Friedman et al. (2012) Tumor-Specific CD4+ Melanoma Tumor-Infiltrating Lymphocytes. J Immunother 35:(5):400-408.
Gabizon et al. (2003) Pharmacokinetics of Pegylated Liposomal Doxorubicin. Clin Pharmacokinet 42(5):419-436.
Gennaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Gross & Mienhofer (eds.) (1981) The Peptides, Vol. 3, Academic Press, New York, New York, United States of America. pages 3-88.
Grossman & Altieri (2001) Drug Resistance in Melanoma: Mechanisms, Apoptosis, and New Potential Therapeutic Targets. Cancer Metastasis Rev 20(1):3-11.
Harlow & Lane (1988) Antibodies, A Laboratory Manual, Cold Spring Harbor Publications, Cold Spring Harbor, New York, United States of America.
Hu et al. (2015) Long-Term Outcomes of Helper Peptide Vaccination for Metastatic Melanoma. Ann Surg 262(3):456-464.
Hunder et al. (2008) Treatment of Metastatic Melanoma with Autologous CD4+ T Cells against NY-ESO-1. N Engl J Med 358(25):2698-2703.
Huston et al. (1988) Protein engineering of antibody binding sites: recovery of specific activity in an anti-digoxin single-chain Fv analogue produced in Escherichia coli. Proc Natl Acad Sci USA 85:5879.
Jones et al (1986) Replacing the complementarity-determining regions in a human antibody with those from a mouse. Nature 321:522.
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
Kelly et al. (2011) Targeted Liposomal Drug Delivery to Monocytes and Macrophages. J Drug Deliv 2011:727241.
Kitano et al. (2013) Enhancement of Tumor-Reactive Cytotoxic CD4+ T Cell Responses after Ipilimumab Treatment in Four Advanced Melanoma Patients. Cancer Immunol Res 1(4):235-244.
La Porta (2007) Drug Resistance in Melanoma: New Perspectives. Curr Med Chem 14(4):387-391.
Li et al. (2018) Nanoliposome C6-Ceramide Increases the Anti-Tumor Immune Response and Slows Growth of Liver Tumors in Mice. Gastroenterology 154(4):1024-1036.e9.
Lim et al. (2019) Current Immunotherapy Approaches for Malignant Melanoma. BioChip J 13 (1):105-114.
Livingston et al. (1985) The Serologic Response to Meth A Sarcoma Vaccines after Cyclophosphamide Treatment Is Additionally Increased by Various Adjuvants. J Immunol 135(2):1505-1509.
Lo et al. (2021) Epitope Spreading toward Wild-Type Melanocyte-Lineage Antigens Rescues Suboptimal Immune Checkpoint Blockade Responses. Sci Transl Med 13(581):eabd8636.
Mata-Haro et al. (2007) The Vaccine Adjuvant Monophosphoryl Lipid A as a TRIF-Biased Agonist of TLR4. Science 316(5831):1628-1632.
Melssen et al. (2019) A Multipeptide Vaccine plus Toll-like Receptor Agonists LPS or PolyICLC in Combination with Incomplete Freund's Adjuvant in Melanoma Patients. J. Immunother. Cancer 7:163.
Nisini et al. (2018) The Multirole of Liposomes in Therapy and Prevention of Infectious Diseases. Front Immunol 9:155.
Pacheco et al. (2020) Nano COVID-19 Vaccines: The Firsts RNA Lipid Nanoparticle Vaccines Being Approved from History—Review. Res Soc Dev 9(12):e20191211123-e20191211123.
Perez Socas & Ambroggio (2020) The influence of myristoylation, liposome surface charge and nucleic acid interaction in the partition properties of HIV-1 Gag-N-terminal peptides to membranes. Biochim Biophys Acta BBA—Biomembr 1862(11):183421.
Ponzoni et al. (2018) Targeting Macrophages as a Potential Therapeutic Intervention: Impact on Inflammatory Diseases and Cancer. Int J Mol Sci 19(7):E1953.
Raetz et al. (2006) Kdo2-Lipid A of Escherichia coli, a Defined Endotoxin That Activates Macrophages via TLR-4. J Lipid Res 47(5):1097-1111.
Riechmann et al. (1988) Reshaping human antibodies for therapy. Nature 332(6162):323-327.
Rudmann (2013) On-Target and Off-Target-Based Toxicologic Effects. Toxicol Pathol 41(2):310-314.
Saremi et al. (2018) The Role of Nanoliposome Bilayer Composition Containing Soluble Leishmania Antigen on Maturation and Activation of Dendritic Cells. Iran J Basic Med Sci 21(5):536-545.
Sasaki & White (2008) Aggregation Behavior of an Ultra-Pure Lipopolysaccharide That Stimulates TLR-4 Receptors. Biophys J 95(2):986-993.
Shaw et al. (2020) Inhibition of Lysosomal Function Mitigates Protective Mitophagy and Augments Ceramide Nanoliposome-Induced Cell Death in Head and Neck Squamous Cell Carcinoma. Mol Cancer Ther 19(12):2621-2633.
Shin et al. (2018) Liposomal Delivery of Diacylglycerol Lipase-Beta Inhibitors to Macrophages Dramatically Enhances Selectivity and Efficacy in Vivo. Mol Pharm 15(3):721-728.
Siegel et al. (2021) Cancer Statistics, 2021. CA Cancer J Clin 71(1):7-33.
Sims et al. (2010) Kdo2-Lipid A, a TLR4-Specific Agonist, Induces de Novo Sphingolipid Biosynthesis in RAW264.7 Macrophages, Which Is Essential for Induction of Autophagy. J Biol Chem 285(49):38568-38579.
Slingluff et al. (2008a) Evaluation of the Sentinel Immunized Node for Immune Monitoring of Cancer Vaccines. Ann Surg Oncol 15(12):3538-3549.
Slingluff et al. (2008b) Helper T-Cell Responses and Clinical Activity of a Melanoma Vaccine With Multiple Peptides From MAGE and Melanocytic Differentiation Antigens. J Clin Oncol 26(30):4973-4980.
Slingluff et al. (2010) Immunogenicity for CD8+ and CD4+ T Cells of Two Formulations of an Incomplete Freund's Adjuvant for Multipeptide Melanoma Vaccines. J Immunother 33:(6):630-638.
Slingluff et al. (2021) Trial to Evaluate the Immunogenicity and Safety of a Melanoma Helper Peptide Vaccine plus Incomplete Freund's Adjuvant, Cyclophosphamide, and PolyICLC (Mel63). J Immunother Cancer 9(1):e000934.
Stolk et al. (2020) Lipo-Based Vaccines as an Approach to Target Dendritic Cells for Induction of T- and iNKT Cell Responses. Front Immunol 11:990.
Suri (2006) Cancer Testis Antigens—Their Importance in Immunotherapy and in the Early Detection of Cancer. Expert Opin Biol Ther 6(4):379-389.
Tassa et al. (2010) Binding Affinity and Kinetic Analysis of Targeted Small Molecule-Modified Nanoparticles. Bioconjug Chem 21(1):14-19.
Tiet & Berlin (2017) Exploiting Homing Abilities of Cell Carriers: Targeted Delivery of Nanoparticles for Cancer Therapy. Biochem Pharmacol 145:18-26.
U.S. Patent Application Publication Nos. 2003/0017534, 2018/0298087, 2018/0312588, 2018/0346564, 2019/0151448.
U.S. Pat. Nos. 4,816,567; 5,482,856; 6,479,284; 6,677,436; 7,060,808; 7,906,625; 8,398,980; 8,436,150; 8,796,439; 10,253,111.
Wang et al. (2015) Kdo2-Lipid A: Structural Diversity and Impact on Immunopharmacology. Biol Rev 90(2):408-427.
Wang et al. (2020) Tet2-mediated clonal hematopoiesis in nonconditioned mice accelerates age-associated cardiac dysfunction. JCI Insight 5:e135204.
Winder & Virós (2018) Mechanisms of Drug Resistance in Melanoma. In Mechanisms of Drug Resistance in Cancer Therapy. Handbook of Experimental Pharmacology, volume 249. Mandalà & Romano (eds.). Springer International Publishing, New York, New York. pp. 91-108.
Winter & Milstein (1991) Man-made antibodies. Nature 349(6307):293-299.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising a stable nanoliposome containing one or more TLR4 agonists, optionally wherein at least one of the one or more TLR4 agonists is 3-deoxy-d-manno-octulosonic acid-lipid A (KDO2-lipid A or KDO2).

2. The composition of claim 1, wherein the KDO2-containing nanoliposome comprises a lipid component comprising, consisting essentially of, or consisting of one or more of, optionally two or more of, and further optionally all three of 1,2-distearoyl-sn-glycero-3-phosphocholine (DSPC); 1,2-Dioleoyl-sn-glycero-3-phosphoethanolamine (DOPE); and 1,2-distearoyl-sn-glycero-3-phosphoethanolamine-N-[methoxy(polyethylene glycol)-2000] (ammonium salt) (PEG2000PE), and optionally further comprises, consists essentially of, or consists of one or more of:
(i) a toll-like-receptor 4 (TLR4) agonist, optionally 3-deoxy-d-manno-octulosonic acid-lipid A (KDO2-lipid A or just KDO2); and/or
(ii) a rigidity enhancer and/or leakiness reducer, optionally cholesterol; and/or
(iii) a dye, optionally a fluorophore, further optionally rhodamine or 1,1′-Dioctadecyl-3,3,3′,3′-Tetramethylindodicarbocyanine, 4-Chlorobenzenesulfonate Salt (DiD); and/or
(iv) optionally 1,2-dioleoyl-3-trimethylammonium-propane (chloride salt) (DOTAP), dihexadecyl phosphate (DHP), or a combination thereof,
or any combination thereof.

3. The composition of claim 1, wherein the KDO2-containing nanoliposome is a neutral liposome comprising a lipid component comprising, consisting essentially of, of consisting of DSPC, DOPE, PEG(2000)-PE, KDO2, Cholesterol, and Rhodamine or DiD at a molar ratio of about 4.6:1.14:0.25:0.01:3.00:0.02, optionally in a buffer comprising about 1.33 mg/ml sodium bicarbonate in a 1:2 solution of Lactated Ringers solution (LR) and water.

4. The composition of claim 1, wherein the KDO2-containing nanoliposome is a cationic liposome comprising a lipid component comprising, consisting essentially of, of consisting of DSPC, DOPE, PEG(2000)-PE, KDO2, Cholesterol, Rhodamine or DiD, and DOTAP at a molar ratio of about 4.12:1.90:0.25:0.01:3.00:0.02:0.70, optionally in a buffer comprising about 5 mg/ml sodium bicarbonate in water.

5. The composition of claim 1, wherein the KDO2-containing nanoliposome is an anionic liposome comprising a lipid component comprising, consisting essentially of, of consisting of DSPC, DOPE, PEG(2000)-PE, KDO2, Cholesterol, Rhodamine or DiD, and DHP at a molar ratio of about 3.91:1.81:0.25:0.01:3.00:0.02:1.00, optionally in a buffer comprising about 1 ml/ml sodium bicarbonate in water or a buffer comprising a 1:9 ratio of 2-(N-morpholino) methanesulfonic acid (MES) buffer and water.

6. The composition of claim 1, wherein the KDO2-containing nanoliposome encapsulates an immunogenic peptide.

7. The composition of claim 6, wherein the immunogenic peptide is a peptide associated with malignant melanoma.

8. The composition of claim 7, wherein the peptide comprises, consists essentially of, or consists of an amino acid sequence that is a subsequence of a protein selected from the group consisting of tyrosinase, gp100, MAGE-1,2,3,6, Melan-A/MART-1, and MAGE-3.

9. The composition of claim 8, wherein:
(i) the tyrosinase peptide comprises, consists essentially of, or consists of amino acids 56-70 (SEQ ID NO: 6) and/or 386-406 (SEQ ID NO: 3) of human tyrosinase; and/or
(ii) the gp100 peptide comprises, consists essentially of, or consists of amino acids 44-59 (SEQ ID NO: 5) of human gp100; and/or
(iii) the MAGE-1,2,3,6 peptide comprises, consists essentially of, or consists of amino acids 121-134 (SEQ ID NO: 2) of human MAGE-1,2,3,6; and/or
(iv) the Melan-A/MART-1 peptide comprises, consists essentially of, or consists of amino acids 51-73 (SEQ ID NO: 4) of human Melan-A/MART-1; and/or
(v) the MAGE-3 peptide comprises, consists essentially of, or consists of amino acids 281-295 (SEQ ID NO: 1) of human MAGE-3.

10. The composition of claim 9, wherein the composition comprises 1, 2, 3, 4, 5, or 6 different peptides selected from the group consisting of a tyrosinase peptide, a gp100 peptide, a MAGE-1,2,3,6 peptide, a Melan-A/MART-1 peptide, and a MAGE-3 peptide, optionally wherein the tyrosinase peptide comprises, consists essentially of, or consists of SEQ ID NO: 3 or SEQ ID NO: 6; the gp100 peptide comprises, consists essentially of, or consists of SEQ ID NO: 5; the MAGE-1,2,3,6 peptide comprises, consists essentially of, or consists of SEQ ID NO: 2; the Melan-A/MART-1 peptide comprises, consists essentially of, or consists of SEQ ID NO: 4; and a MAGE-3 peptide comprises, consists essentially of, or consists of SEQ ID NO: 1.

11. The composition of claim 1, wherein the composition is a pharmaceutical composition, optionally a pharmaceutical composition that is pharmaceutically acceptable for use in a mammal, further optionally wherein the mammal is a human.

12. The composition of claim 1, further comprising at least one adjuvant.

13. The composition of claim 12, wherein the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

14. A method for treating and/or preventing malignant melanoma, the method comprising administering to a subject in need thereof an effective amount of the composition of claim 1.

15. The method of claim 14, wherein the subject in need thereof is a human.

16. A method for inducing an anti-melanoma immune response in a subject, the method comprising administering to the subject an effective amount of the composition of claim 1.

17. The method of claim 16, wherein the subject in need thereof is a human.

18. The method of claim 16, wherein the administering is via a route selected from the group consisting of intravenous and subcutaneous.

19-22. (canceled)
